(19) United States
(12) Patent Application Publication
(10) Pub. No.: US 2005/0163866 A1
(43) Pub. Date: Jul. 28, 2005

(54) Solid dosage form

(76) Inventors: Rainer Alex, Weil am Rhein (DE); Benno Rothenhaeusler, Loerrach (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/037,905

(22) Filed: Jan. 18, 2005

(30) Foreign Application Priority Data
Jan. 22, 2004 (EP) 04001328.6

(51) Int. Cl. A61K 33/26; A61K 49/00

(52) U.S. Cl. 424/646; 424/9.36

(57) ABSTRACT

A solid dosage form (1) which comprises a predefined magnetic pattern (2), representing information. The dosage form (1) may be an oral dosage form such as a tablet or a capsule, and the magnetic pattern (2) may be invisible. The information contained in the magnetic pattern (2) may be coded.

### FIELD OF THE INVENTION

The present invention relates to a solid dosage form having a predefined magnetic pattern useful for tracking the dosage form and preventing counterfeiting.

### BACKGROUND OF THE INVENTION

In the pharmaceutical industry, counterfeit protection has become an important feature of drug manufacture. Depending on the country—considerable amounts of counterfeited, illegal medicaments have been found to have penetrated the market. This could have severe consequences for patients, especially in the case of life-supporting medicaments. Therefore, regulatory authorities in charge of admission of medicaments to the respective domestic markets are placing more of the burden of counterfeit protection on manufacturers.

While the imprinting of logos of the manufacturer company into the outer surface of a tablet is a conventional measure to identify the origin of the tablet, the level of protection against counterfeiting provided by this measure is very low. On the other hand, suppliers (such as pharmacies, physicians, etc.) want to be sure or need to be sure (e.g. for liability reasons), that they are distributing to their consumers the original product from a specific manufacturer.

Also, the identity of a solid dosage form must be ensured and documented throughout the whole manufacturing process and during storage and distribution. This is usually performed in a non-destructive way by assessing characteristic, visible features of the dosage form like imprint, color or shape. These features can either be assessed by a human or read by a machine. However, it is at least difficult if not impossible to assess or read these features without establishing mechanical contact between the dosage form and the reader except when the dosage form is exposed to a suitable external source of light or radiation. Also, quality control cannot easily be performed.

### SUMMARY OF THE INVENTION

Accordingly, it is an object of the invention to provide a solid dosage form that overcomes the aforementioned disadvantages.

This object is achieved with the solid dosage form comprising a predefined magnetic pattern.

In one embodiment the present invention provides a solid dosage form comprising a predefined magnetic pattern representing information.

Although U.S. Pat. No. 5,079,006 discloses a pharmaceutical solid dosage form that contains magnetic material, it does not contain a predefined magnetic pattern and does not produce a detectable magnetic signal, even when suitable detectors are available.

A magnetic pattern may not be easily detected by potential counterfeiters, and even where it is detected by a counterfeiter it is difficult to copy. On the other hand, a suitable magnetic pattern is comparatively easy to manufacture. Also, with a suitable means, suppliers (e.g. pharmacies, physicians, etc.) can easily verify whether a dosage form is the original product that they distribute to consumers. Also, the magnetic pattern allows quality control as well as assessment of the identity of the dosage form without a need for external sources of light or radiation.

### BRIEF DESCRIPTION OF THE FIGURES

FIG. 1 shows an embodiment of a solid dosage form carrying a magnetic pattern representing information, in accordance with the instant invention,

FIG. 2 shows an embodiment of an arrangement for producing the magnetic pattern contained in the embodiment of the dosage form of FIG. 1.

### DETAILED DESCRIPTION OF THE INVENTION

Solid dosage forms are conventional dosage forms that are well accepted around the whole globe. While the term “solid dosage form” within this specification is intended to cover all types of solid dosage forms (e.g. tablets, capsules suppositories, etc.) and all types of applications (e.g. cosmetic, pharmaceutical, diagnostic, nutritional, dietary etc. applications), solid dosage forms of particular interest are oral dosage forms containing a pharmaceutically active ingredient, a cosmetic ingredient, a diagnostic reagent, or a nutritional or dietary supplement, etc.

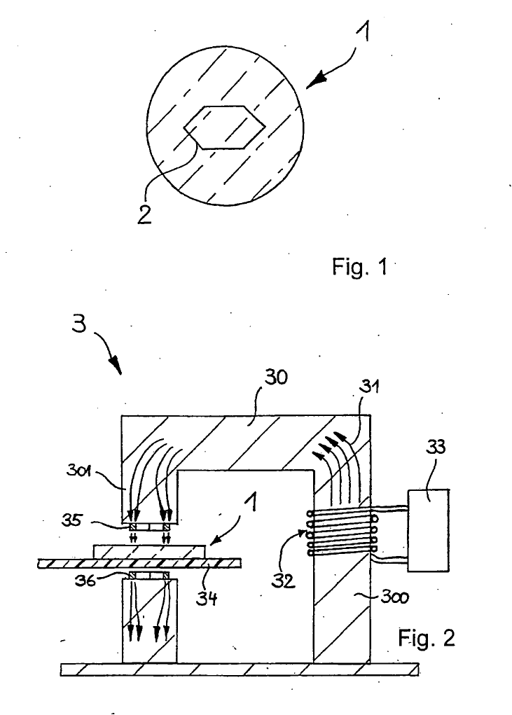

A “predefined magnetic pattern” is a magnetic pattern which is produced in a controlled magnetization process, e.g. by selective magnetization of portions of magnetic material, e.g. as illustrated in FIGS. 1 and 2. It is characterized by a spatial distribution of magnetized material.

The term “magnetization process” refers to a physical process where a net magnetization is produced in a magnetic material or substance by a given magnetic field. A magnetic material is thereby transformed into a magnetized material.

The term “magnetic material” refers to any material that shows some magnetic property. All known elements show some magnetic property. Therefore, all known materials are magnetic materials. The most common magnetic properties are diamagnetism, paramagnetism and ferromagnetism. Magnetic materials may appear unmagnetized as a whole, that is, they may have no net magnetization on their own. Magnetic materials do not automatically generate magnetic fields.

A “magnetized material” is produced from a magnetic material or substance by the physical process of magnetization. A magnetized material or substance has therefore the same chemical composition as the respective magnetic material.

The term “magnetic resonance” refers to the absorption or emission of electromagnetic radiation by electrons or atomic nuclei in response to the application of certain external magnetic fields.

A “magnetic field” is the region in the neighborhood of a magnet, electric current or changing electric field in which magnetic forces are observable. Magnetic fields may be represented mathematically by quantities called vectors.

The term “magnet” refers to a material capable of attracting iron and producing a magnetic field outside itself. A magnet is produced from a magnetic material by the process of magnetization. For example, iron, a ferromagnetic material, is usually not capable of attracting other items made from iron unless it has been magnetized.

A preferred solid dosage form of the invention may be a pharmaceutically active, cosmetic, diagnostic, nutritional, dietary, etc., dosage form, in particular an oral dosage form (such as a tablet, e.g. coated tablet, multi-layer tablet, dragee, pill, granules, powder, or capsule) containing a pharmaceutically active ingredient, a cosmetic ingredient, a diagnostic reagent, a nutritional or dietary supplement, etc. (this is meant to also include any combination of such ingredients). These types of dosage forms are widespread and well accepted by consumers.

The solid dosage form may comprise a core carrying the magnetic pattern and at least one coating covering the core. In particular, the coating covering the core may be opaque, so that the magnetic pattern carried by the core is undetectable (e.g. invisible) by a human without any specific detection (e.g. visualization) means.

Alternatively, the coating, rather than the core, may carry the magnetic pattern. In this case, the magnetic pattern may be detectable or undetectable by a human. If necessary, a further coating may be provided which may be opaque in order to ensure that the magnetic pattern is undetectable (e.g. invisible).

In one embodiment the solid dosage form may also comprise a separately manufactured film affixed to the dosage form, where the film carries the magnetic pattern. This embodiment allows the separate manufacture of the raw dosage form and of the film carrying the magnetic pattern. The film is then affixed to the raw dosage form to produce the final dosage form. One type of film useful in the invention is a polymer film that is separately manufactured and then attached to the raw dosage form. Again, the magnetic pattern contained in the film may be detectable (e.g. visible) or may be undetectable (e.g. invisible) by a human (e.g. it may be visible only with a specific visualization means).

The information contained in the magnetic pattern is advantageously undetectable by a human without any separate detection (e.g. visualization) means. For example, the magnetic pattern can be provided in the interior of the dosage form so that a potential counterfeiter is prima facie unable to recognize that there is any protection against counterfeiting at all contained in the dosage form.

The information may be contained in the magnetic pattern in any form. For example, it can be a simple representation of the manufacturer's logo. However, it may be more advantageous if the information contained in the magnetic pattern is encoded. Encoded information raises the level of protection against counterfeiting, since it may not be easy for the counterfeiter to decode the information contained in the code, making counterfeiting even more difficult.

The magnetic pattern of the solid dosage form according to the invention is provided by a physiologically acceptable magnetized ancillary substance. The physiologically acceptable magnetized ancillary substance ensures that the magnetic pattern is not detrimental to the consumer.

In particular, the physiologically acceptable magnetized ancillary substance is selected from iron (Fe), iron-II-oxide (Fe₂O₃) or iron-III-oxide (Fe₃O₄). These ancillary substances are known as being physiologically acceptable and magnetizable so as to form the magnetic pattern.

Further advantageous features of the invention will become apparent from the following detailed description of an embodiment of the invention with the aid of the drawings.

FIG. 1 shows an embodiment of a solid dosage form according to the instant invention in the form of a tablet 1 comprising a pharmaceutically active ingredient, although the tablet does not mandatorily contain a pharmaceutically active ingredient but instead may contain other active ingredients, e.g. vitamins or ingredients for obtaining cosmetic effects, diagnostic ingredients, nutritional or dietary ingredients, etc., or may contain combinations of different types of active ingredients. Tablet 1 comprises a magnetic pattern 2. Magnetic pattern 2 has the form of a specific hexagon which may constitute a part of the logo of a specific manufacturer (such as applicant's logo). A logo helps to identify the manufacturer of tablet 1 but does not represent a sophisticated code.

Therefore, as already described hereinbefore, magnetic pattern 2 may appear in the form of a more sophisticated code, such as a bar code. In this case, the bar code may include information (e.g. information about the manufacturer, the name of the medicament, a production lot number, the production date, etc.) which makes it considerably more difficult, and maybe impossible, for counterfeiters to exactly copy the bar code. As a further alternative, the logo may comprise different precisely located points on the logo which generate a magnetic field that is considerably stronger than the field generated by the remainder of points located on the logo. At these precisely located points, the magnetic field is stronger than a predefined threshold value. Such a specific code is practically impossible to be detected by a counterfeiter, but during verification of the final product to be distributed to the consumer, suppliers (e.g. pharmacies) can easily verify that the product they distribute to consumers is the original product rather than a counterfeit. This can be done, for example, with the aid of a suitable device (detecting the magnetic pattern and comparing the field strength at the precisely located points with the threshold value).

As is illustrated in FIG. 1 by the hatching, magnetic pattern 2 may be invisible (i.e. undetectable) to a human from outside tablet 1 without the use of a specific visualization (i.e. detection) means. In order to achieve this, magnetic pattern 2 may be contained in the core of tablet 1. The core may be covered by at least one opaque coating (not shown in FIG. 1) in order to render magnetic pattern 2 invisible.

Alternatively, magnetic pattern 2 may be contained in the coating. The coating may be opaque so that magnetic pattern 2 is invisible to a human. In case the coating containing magnetic pattern 2 is not opaque the coating itself may be covered by an additional opaque coating in order to ensure that magnetic pattern 2 is invisible.

Furthermore, magnetic pattern 2 may be contained in a film that can be separately manufactured, e.g. a polymer film (e.g. a polymer film containing a magnetizable ancillary substance), which is then affixed (e.g. bonded) to the raw tablet in order to form the final tablet 1.

Magnetic pattern 2 can be provided within tablet 1 by at least one physiologically acceptable magnetized anciallary substance, such as for example iron (Fe), iron-II-oxide (Fe₂O₃) or iron-III-oxide (Fe₃O₄). These ancillary substances are known as being physiologically acceptable.

Magnetic pattern 2 can also be produced by printing the pattern onto the core or onto the coating using a magnetizable, physiologically acceptable ink and then drying the ink. The ink can be applied in a magnetic field so that the magnetizable particles contained in the ink are immediately magnetized, or the ink can be applied and dried and then magnetization is performed, for example with the aid of a movable writing head generating a suitable magnetic field. Also, a visible (non-magnetic) pattern may be provided carrying an overlaid invisible magnetic pattern.

One embodiment of an arrangement 3 for producing the magnetic pattern contained in tablet 1 shown in FIG. 1 is represented in FIG. 2. Arrangement 3 comprises an iron core 30 for guiding a magnetic flux 31 that is generated by a winding 32 arranged on a leg 300 of iron core 30. Winding 32 is supplied with an electric current by a power supply 33 to which it is connected. The electric current flowing through winding 32 produces magnetic flux 31 represented by the respective arrows shown in FIG. 2. In an air gap provided in a further leg 301 of iron core 30 there is arranged a non-magnetic carrier 34 that carries tablet 1. The dimensions of the air gap are represented highly exaggerated in FIG. 2 for clearness reasons. For the sake of simplicity let us assume, that a magnetizable ancillary substance is already provided in tablet 1 but has not yet been magnetized. Upon switching on power supply 33, magnetic flux 31 is generated and guided through iron core 30. On both sides of tablet 1 or carrier 34, respectively, there are arranged templates 35 and 36 corresponding to the shape of magnetic pattern 2 shown in FIG. 1. Since templates 35 and 36 are made from iron (or a material having a high magnetic permeability μ when compared to air) essentially the whole magnetic flux is guided through templates 35 and 36. As a consequence, the magnetizable ancillary substance is magnetized to form magnetic pattern 2 having the shape as shown in FIG. 1.

As has been described above, instead of providing fixedly arranged templates 35 and 36, a moveable writing head can be moved along the contour of the logo so as to produce magnetic pattern 2. Also, other methods (see further above) may be applied to generate magnetic pattern 2.

Magnetic pattern 2 contained in tablet 1 can be detected by any suitable means. Recently, thin transparent magneto-optical films have become available for the direct visualization of magnetic fields (e.g. a film offered under the trade name Kel-View™, available from Kelvin, Inc., USA).

Other detection systems are also contemplated which are based on the principle of electromagnetic induction. For example, when the tablet is moved relative to a sensor of the detection system, the “magnet” (the tablet) in motion produces a time varying magnetic field that induces an electric current in the sensor. Sensors of this type are already used, for example, in tape recorders or card readers. Other types of detection systems utilize the so-called “Hall-effect”, that is to say an electric current passing through a conductor is modified by a static magnetic field (the magnetic field acts upon the moving electrons in the conductor). Such detection systems are already used, for example, for counterfeit detection of checks. Further types of detection systems comprise hand-held pens for reading out magnetic information (e.g. of bar codes etc.). Such detection systems are commercially available from a plurality of manufacturers, for example from Stopfraud, Inc., Atlanta, USA). The electric signals produced by the aforedescribed detection systems may be converted so that a respective image can be produced on a conventional screen.

As can be seen, the solid dosage form according to the invention offers a measure (providing a magnetic pattern) that is simple to manufacture but difficult to counterfeit. While the embodiments described above are intended to show examples of the invention, the scope of protection is intended to be defined by the appended claims.

1. Solid dosage form (1) comprising a predefined magnetic pattern (2).

2. Solid dosage form (1) according to claim 1, comprising a predefined magnetic pattern (2) representing information.

3. Solid dosage form (1) according to claim 1, wherein the magnetic pattern (2) is provided by at least one physiologically acceptable magnetized ancillary substance.

4. Solid dosage form (1) according to claim 1, wherein the solid dosage form (1) is a pharmaceutically active, cosmetic, diagnostic or dietary dosage form.

5. Solid dosage form (1) according to claim 1, wherein the solid dosage form (1) is an oral dosage form.

6. Solid dosage form (1) according to claim 5, wherein the oral dosage form is a tablet or a capsule.

7. Solid dosage form (1) according to claim 2, wherein the information contained in the magnetic pattern (2) is undetectable by a human without any separate detection means.

8. Solid dosage form (1) according to claim 2, wherein the information contained in the magnetic pattern (2) is encoded.

9. Solid dosage form (1) according to claim 3, wherein the physiologically acceptable magnetized ancillary substance is selected from iron (Fe), iron-II-oxide (Fe₂O₃) or iron-III-oxide (Fe₃O₄).

10. Solid dosage form (1) according to claim 1, wherein the dosage form (1) comprises a core carrying the magnetic pattern (2) and at least one coating covering the core.

11. Solid dosage form (1) according to claim 10, wherein the coating covering the core is opaque.

12. Solid dosage form (1) according to claim 1, wherein the dosage form comprises a core and at least one coating covering the core, the coating carrying the magnetic pattern (2).

13. Solid dosage form (1) according to claim 1, comprising a separately manufactured film affixed to the dosage form, the film carrying the magnetic pattern (2).
